(19) United States
(12) Patent Application Publication
(10) Pub. No.: US 2008/0038328 A1
(43) Pub. Date: Feb. 14, 2008

(54) Pasting Preparation

(76) Inventors: Naruhito Higo, Saga (JP); Tetsuro Tateishi, Ibaraki (JP); Takaaki Terahara, Ibaraki (JP)

Correspondence Address: NATH & ASSOCIATES, 112 South West Street, Alexandria, VA, 22314, US

(21) Appl. No.: 11/597,517

(22) PCT Filed: May 30, 2005
(86) PCT No.: PCT/JP05/09877

§ 371 Date: Feb. 9, 2007

(30) Foreign Application Priority Data
May 28, 2004 (JP) 2004-159846

(51) Int. Cl. A61K 9/70; A61K 31/216; A61K 31/445; A61K 31/48; A61K 47/12; A61K 47/30

(52) U.S. Cl. 424/448; 514/284; 514/329; 514/534; 514/772.3; 514/784

(57) ABSTRACT

The patch preparation of the present invention is a patch preparation comprising a backing and an adhesive layer provided on the backing; wherein, the adhesive layer contains (A) a volatile organic acid and (B) a basic drug; the molar concentration ratio [(M_{A})/(M_{B})] between the molar concentration (M_{A}) of the component (A) and the molar concentration (M_{B}) of the component (B) in the adhesive layer is 0.5 or more; and the component (B) contains a basic drug formed as an ion pair with an anion component.

### TECHNICAL FIELD

The present invention relates to a patch preparation.

### BACKGROUND ART

Various methods have conventionally been used to administer drugs, examples of which include oral administration, rectal administration, intracutaneous administration and intravenous administration, and oral administration is used particularly commonly. In the case of oral administration, however, there were disadvantages such as being susceptible to first pass effects in the liver following absorption of the drug, blood concentrations being temporarily observed beyond that which is necessary following administration. In addition, numerous adverse side effects have also been reported for oral administration, such as gastroenterological disorders, nausea and loss of appetite. In addition, there has recently been a growing clinical desire for preparations that can be taken more easily in consideration of the increasing number of patients having decreasing swallowing ability accompanying the growing size of the elderly population. Thus, development of preparations capable of being administered percutaneously has proceeded aggressively and several products have been commercialized for the purpose of eliminating the shortcomings of oral administration by allowing preparations to be taken easily by patients both safely and continuously.

However, there are many cases in which percutaneous drug absorption is inadequate in these percutaneously absorbed drugs, and it is difficult to develop many of these drugs into percutaneously absorbed preparations due to the low percutaneous absorption thereof, thereby preventing this objective from being achieved. Namely, since normal skin inherently has a barrier function for preventing the entrance of foreign objects therein, there are many cases in which it is difficult for pharmacologically active ingredients blended therein to be adequately absorbed through the skin in the case of bases used in ordinary percutaneously absorbed preparations.

Consequently, patch preparations have been previously reported in which a drug is blended with an organic acid and/or organic acid salt for the purpose of improving percutaneous absorption of a drug through the horny layer of the skin (for example, see Patent document 1-6). These patch preparations attempt to improve skin permeability of a drug by combining the drug with an organic acid and/or organic acid salt.
Patent document 1: Japanese Laid-Open Patent Application No. H10-45570.
Patent document 2: Japanese Laid-Open Patent Application No. H11-302161.
Patent document 3: International Publication WO 00/61120.
Patent document 4: International Publication WO 01/007018.
Patent document 5: International Publication WO 01/005381.
Patent document 6: International Publication WO 02/038139.

### DISCLOSURE OF THE INVENTION

### Problems to be Solved by the Invention

However, examples of typical methods for producing patch preparations include: (1) a method in which a plastic containing a pharmacologically active ingredient is applied on a backing or release sheet to form an adhesive layer followed by laminating a release sheet or backing to the adhesive layer (this method may be referred to as the “plaster application method” in the present specification), (2) a method in which adhesive base containing a pharmaceutically active ingredient and a solvent is coated onto a backing or release sheet, an adhesive layer is formed by drying the coated film until the solvent is removed from the formed coated film, and laminating a release sheet or backing onto the adhesive layer (this method may be referred to as the “solvent method” in the present specification), and (3) a method in which a pharmacologically active ingredient is added to a resin composition melted at a high temperature, an adhesive layer is formed by spreading this onto a backing or release sheet, and laminating a release sheet or backing onto the adhesive layer (this method may be referred to as the “hot melt method” in the present specification).

However, in the case of producing any of the patch preparations described in Patent document 1-6 using the methods described above, the percutaneous drug absorption of the resulting patch preparation differed depending on the production method. In particular, there were cases in which the desired percutaneous drug absorption was unable to be obtained in the case of using the solvent method or hot melt method. Consequently, there is still room for improvement in various aspects of these production methods with respect to the above-mentioned patch preparations of the prior art.

Thus, with the foregoing in view, an object of the present invention is to provide a patch preparation having superior percutaneous absorption of drug even in the case of being produced using ordinary patch preparation production methods.

### Means for Solving the Problems

As a result of conducting studies on patch preparations provided with an adhesive layer formed from an adhesive base containing a drug and an organic acid and/or organic acid salt, the inventors of the present invention found that an adhesive layer comprised of a basic drug and a volatile organic acid results in superior percutaneous absorption of drug.

Moreover, as a result of conducting further studies on the basis of the above finding, the inventors of the present invention found that, by using a specific value for the concentration ratio between a basic drug and a volatile organic acid contained in an adhesive layer, a basic drug forming an anion component and ion pair, can be adequately incorporated in the adhesive layer, and as a result, the skin permeation rate of the drug is increased, and by increasing the concentration of the volatile organic acid, the skin permeation rate of the drug can be increased considerably, thereby leading to completion of the present invention.

Namely, a patch preparation of the present invention comprises a backing and an adhesive layer provided on the backing; wherein the adhesive layer contains a volatile organic acid (A) and a basic drug (B), and wherein in adhesive layer the molar concentration ratio [(M_{A})/(M_{B})] between the molar concentration (M_{A}) of the component (A) and the molar concentration (M_{B}) of the component (B) is 0.5 or more, and the component (B) contains a basic drug formed as an ion pair with an anion component.

Here, the component (A) also contains a volatile organic acid present in the form of an anion component of the basic drug formed as an ion pair with an anion component.

According to the present invention, by making the molar concentration ratio [(M_{A})/(M_{B})] between the molar concentration (M_{A}) of the component (A) and the molar concentration (M_{B}) of the component (B) 0.5 or more, the content of the basic drug formed as an ion pair with an anion component can be made to be of an adequate level, thereby making it possible to realize a patch preparation have adequately superior drug percutaneous absorption.

In addition, according to the present invention, since the conditions for obtaining the above-mentioned effects are specified by the molar ratio between the component (A) and the component (B) in the adhesive layer, adequate percutaneous absorption can be reliably obtained regardless of the production method of the patch preparation. Namely, in the solvent method, by measuring the amount of volatile organic acid that volatizes from the time the adhesive base is prepared until the adhesive layer is formed, and adding the amount lost to the adhesive base in advance, the molar ratio between the component (A) and the component (B) in the adhesive layer can be reliably made to be 0.5 or more. In addition, in other production methods as well, the amount of volatile organic acid lost during production is measured, and blending of plaster is then adjusted on the basis thereof.

In addition, as a result of a volatile organic acid being contained in the adhesive layer under the above-mentioned conditions, in addition to percutaneous absorption of drug being promoted, the effect of improving the drug stability in the adhesive layer, and the effect of alleviating irritation of the skin as a result of the basic drug being neutralized, can be more reliably obtained.

In addition, in the present invention, the above-mentioned molar concentration ratio [(M_{A})/(M_{B})] is preferably 1 or more.

As a result of satisfying these conditions, the content in the adhesive layer of a basic drug formed as an ion pair with an anion component, and particularly a basic drug formed as an ion pair with the above-mentioned volatile organic acid, can be further increased, thereby resulting in even more superior drug percutaneous absorption of the patch preparation.

In addition, in the patch preparation of the present invention, the component (B) may not substantially contain a free form of a basic drug.

In the patch preparation of the present invention, the adhesive layer is preferably formed by removing a solvent from a coated film comprised of an adhesive base containing a volatile organic acid, a basic drug and/or a pharmaceutically acceptable salt of the basic drug.

As a result of forming an adhesive layer using an adhesive base containing a solvent, in addition to being able to form a basic drug formed as an ion pair with an anion component more reliably, the adhesive layer can be made to be more uniform, thereby making it possible to obtain the superior drug percutaneous absorption with greater stability. In addition, in the solvent method, acrylic adhesives and other adhesive bases lacking thermoplasticity ordinarily used as medical adhesives can be used, thereby reducing limitations on the types of adhesive bases able to be used as compared with the hot melt method. Namely, this results in the advantage of offering a higher degree of freedom in the design of the adhesive layer. Moreover, even in cases of incorporating a drug having low thermal stability (and particularly drugs for which problems occur under conditions of subjecting to a temperature of 100° C. for several hours), a patch preparation is more easily obtained which still demonstrates the desired pharmacological effects.

Furthermore, although loss of a volatile component is normally a problem when a patch preparation containing a volatile component is produced according to the solvent method, since the patch preparation of the present invention is composed so as to be unaffected by the loss of volatile component as described above, even in the case of being produced according to the solvent method, the desired superior pharmacological effects are obtained.

In addition, the solvent is preferably one type of solvent selected from the group consisting of toluene, heptane, ethyl acetate, hexane and cyclohexane, or a mixed solvent of two or more types thereof.

In the patch preparation of the present invention, the ratio (SA/SB) of the mass percentage SA of volatile organic acid contained in the adhesive layer to the mass percentage SB of volatile organic acid contained in the adhesive base based on the total mass of all components excluding the solvent in the adhesive base is preferably 0.3 to 0.9. As a result of making this ratio within the range of 0.3 to 0.9, a patch preparation can be realized which has adequately superior productivity as well as adequately superior drug percutaneous absorption.

In the patch preparation of the present invention, the adhesive base preferably also contains an organic acid salt.

As a result of combining the use of an organic acid salt, in addition to further promoting drug percutaneous absorption, the effect is obtained of inhibiting volatilization of the volatile organic acid during formation of the adhesive layer.

In the patch preparation of the present invention, the component (B) preferably contains a basic drug which is formed from an organic acid salt and a salt of a basic drug, as the basic drug formed as an ion pair with an anion component. As a result of containing such a basic drug, the solubility of the drug in the patch preparation is improved, and the effect is obtained of promoting drug transport to the skin based on a concentration difference.

In addition, in the patch preparation of the present invention, the organic acid salt is preferably at least one type selected from the group consisting of sodium acetate, sodium citrate, sodium propionate and sodium lactate.

These organic acid salts are preferable since they are highly safe for the body, and demonstrate low local irritation of the skin in particular. In addition, the effect of inhibiting volatilization of the volatile organic acid during formation of the adhesive layer is obtained more reliably. As a result, the basic drug formed as an ion pair with an anion component (particularly with a volatile organic acid salt) can be formed more efficiently, thereby enabling the realization of a patch preparation having more superior productivity and storage stability.

In the patch preparation of the present invention, the volatile organic acid is preferably at least one type selected from the group consisting of acetic acid, propiohic acid and lactic acid. As a result of using such a volatile organic acid, the content in the adhesive layer of a basic drug formed as an ion pair with an anion component, and particularly a basic drug formed as an ion pair with the volatile organic acid, can be further increased, thereby allowing the effect of improving stability of the drug in the adhesive layer, as well as the effect of alleviating irritation of the skin as a result of neutralizing the basic drug, to be obtained more reliably and easily.

In the patch preparation of the present invention, the basic drug is preferably fentanyl, oxybutynin, pergolide or donepezil. These drugs demonstrate increased skin permeability more reliably as a result of adopting a form in which an ion pair with an anion component (particularly, with a volatile organic acid) is formed in an adhesive layer in which the molar concentration ratio [(M_{A})/(M_{B})] is 0.5 or more. As a result, a patch preparation having adequately superior drug percutaneous absorption can be realized more reliably.

In the patch preparation of the present invention, the adhesive layer preferably contains a water-soluble polymer.

As a result of containing a water-soluble polymer in the adhesive layer, since aqueous components such as perspiration produced by the skin can be absorbed, decreases in adhesive strength and decreases in problems with the feel during use such as moistness can be inhibited, thereby improving the ease of use of the patch preparation.

In addition, the water-soluble polymer is preferably polyvinyl pyrrolidone or a basic nitrogen-containing polymer.

As a result of using polyvinyl pyrrolidone or a basic nitrogen-containing polymer for the water-soluble polymer, together with improving the ease of use of the patch preparation, the physical properties of the preparation can be further improved. In addition, the effect is obtained of inhibiting volatilization of the volatile organic acid during formation of the adhesive layer.

In addition, the basic nitrogen-containing polymer is preferably a methyl methacrylate-butyl methacrylate-dimethylaminoethyl methacrylate copolymer.

As a result of containing such a basic nitrogen-containing polymer, the ease of use of the patch preparation and the physical properties thereof can be further improved.

In addition, the present invention provides a patch preparation comprising a backing, and an adhesive layer provided on the backing and incorporating a volatile organic acid, basic drug and/or pharmaceutically acceptable salt of the basic drug; wherein the total molar concentration of a volatile organic acid and a volatile organic acid derivative soluble in tetrahydrofuran contained in the adhesive layer is 0.5 times or more the molar concentration of the basic drug contained in the adhesive layer.

Furthermore, there are no particular limitations on the form of the basic drug contained in the adhesive layer. In addition, values determined by analysis using high-performance liquid chromatography (HPLC) or gas chromatography can be used for the above-mentioned molar concentrations.

This patch preparation has adequately superior drug percutaneous absorption as a result of being provided with an adhesive layer having the constitution described above. The reason for obtaining such effects is thought to be that, in an adhesive layer which satisfies the above-mentioned conditions, the basic drug formed as an ion pair with an anion component (particularly, with a volatile organic acid) is present in an adequate amount, thereby resulting in an increase in the skin permeation rate of the drug.

In addition, since the patch preparation is defined by the total molar concentration of a volatile organic acid and a volatile organic acid derivative soluble in tetrahydrofuran contained in an adhesive layer, and the molar concentration of a basic drug contained in an adhesive layer, adequate percutaneous absorption can be reliably obtained regardless of the production method of the patch preparation. Namely, in the solvent method, by measuring the amount of volatile organic acid that volatizes from the time the adhesive base is prepared until the adhesive layer is formed, and adding the amount lost to the adhesive base in advance, the total molar concentration of a volatile organic acid and a volatile organic acid derivative soluble in tetrahydrofuran contained in the adhesive layer can be reliably made to be 0.5 times or more the molar concentration of the basic drug contained in the adhesive layer. In addition, in other production methods as well, the amount of volatile organic acid lost during production is measured, and blending of plaster is then adjusted on the basis thereof. In addition, since the volatile organic acid and volatile organic acid derivative (and particularly, a volatile organic acid present in the form of an anion component of a basic drug formed as an ion pair with an anion component) can be extracted from the adhesive layer with tetrahydrofuran, even if the adhesive layer is composed by containing a polymer (such as styrene-isoprene-styrene block copolymer, polybutylene or acrylic polymer), the above-mentioned total molar concentration can be determined with high accuracy, thereby making it possible to reliably realize a patch preparation having adequately superior drug percutaneous absorption.

In addition, the present invention provides a method for increasing the pharmacological effect of a patch preparation provided with a backing and an adhesive layer containing a basic drug provided on the backing; wherein, the adhesive layer is formed from an adhesive composition containing a volatile organic acid, a basic drug and/or a pharmaceutically acceptable salt of the basic drug, the molar concentration of the volatile organic acid (A) is 0.5 times or more the molar concentration of the basic drug (B), and a basic drug formed as an ion pair with an anion component is contained in the adhesive layer.

Here, a volatile organic acid present as an anion component of the basic drug formed as an ion pair with an anion component is also contained in the component (A).

According to this method for increasing the pharmacological effect of a patch preparation of the present invention, by making the molar concentration of the volatile organic acid 0.5 times or more the molar concentration of the basic drug in the adhesive layer, the basic drug formed as an ion pair with an anion component can be contained at an adequate concentration in the adhesive layer, the skin permeation rate of the drug increases due to the action of basic drug formed as an ion pair with an anion component (particularly, with a volatile organic acid), and as a result, the pharmacological effect of the patch preparation can be adequately increased.

In addition, in the method of the present invention, since the molar concentration of the component (A) and the molar concentration of the component (B) are defined in the adhesive layer, the pharmacological effect of the patch preparation can be increased regardless of the method used to form the adhesive layer. Namely, regardless of whether the adhesive layer is formed by a method such as a solvent method or hot melt method, as a result of suitably setting the incorporated amount of volatile organic acid corresponding to the formation method as described above, the effect of increasing the skin permeation rate of the basic drug can be stably obtained.

### EFFECT OF THE INVENTION

According to the present invention, a patch preparation can be provided having adequately superior drug percutaneous absorption even if produced using an ordinary method for producing patch preparations.

### BRIEF DESCRIPTION OF THE DRAWINGS

FIG. 1 is a perspective view showing a preferable embodiment of a patch preparation of the present invention.

### BEST MODE FOR CARRYING OUT THE INVENTION

The following provides a more detailed explanation of preferable embodiments of the present invention with reference to the drawings.

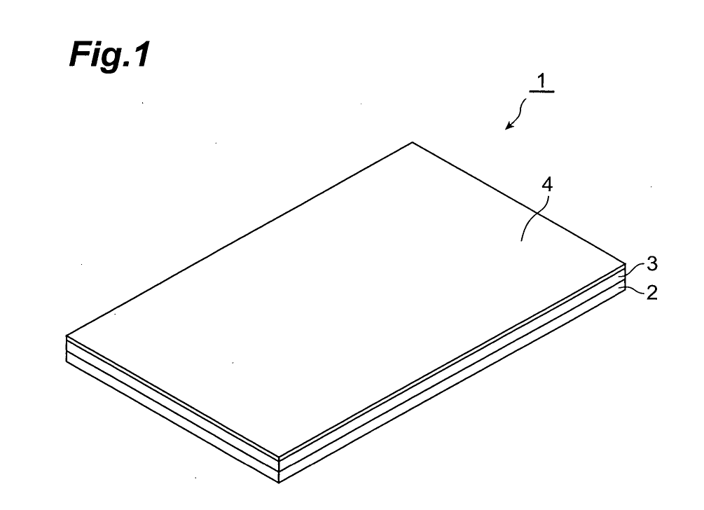

FIG. 1 is a perspective view showing a preferable embodiment of a patch preparation of the present invention. In FIG. 1, a patch preparation 1 is provided with a backing 2, an adhesive layer 3 laminated on the backing 2, and a release sheet 4 adhered on the adhesive layer 3.

The adhesive layer 3 provided in the patch preparation 1 of the present embodiment is formed from an adhesive base containing a volatile organic acid and a basic drug and/or pharmaceutically acceptable salt of a basic drug.

In the present embodiment, the ratio between a volatile organic acid component (A) and a basic drug component (B) present in the adhesive layer 3 is such that the molar concentration ratio [(M_{A})/(M_{B})] of component (A) to component (B) is 0.5 or more, and a basic drug formed as an ion pair with an anion component is contained as component (B) in the adhesive layer 3. As a result of being provided with such an adhesive layer, the percutaneous absorption of the basic drug of patch preparation 1 of the present embodiment is adequately superior. Furthermore, in the present embodiment, the above-mentioned anion component refers to an organic anion derived from an organic acid and/or organic acid salt, examples of which include organic carboxylic acid anion and organic sulfonic acid anion. An organic carboxylic acid anion having 2 to 10 carbon atoms is preferable for the organic carboxylic acid anion, while an acetic acid anion is particularly preferable.

In the present embodiment, although differing according to the type of drug to be described later, the above-mentioned molar concentration ratio [(M_{A})/(M_{B})] is preferably 1 or more. In addition, the upper limit of the molar concentration ratio [(M_{A})/(M_{B})] is preferably 20 or less, and more preferably 5 or less. If the molar concentration ratio [(M_{A})/(M_{B})] exceeds 20, the volatile organic acid tends to bleed from the adhesive layer, thereby tending to cause a decrease in the adhesiveness of the adhesive layer.

Examples of volatile organic acids incorporated in the adhesive base include acetic acid, propionic acid, lactic acid, salicylic acid and derivatives thereof, and benzoic acid. These can be contained alone or two or more types can be contained in combination.

In addition, among these volatile organic acids, acetic acid, propionic acid and lactic acid are preferable, and they can be contained alone or two or more types can be contained in combination.

In consideration of being able to obtain adequate drug percutaneous absorption and irritation of the skin during use as a patch preparation, the content of the volatile organic acid in the adhesive base is preferably set to be 0.1 to 30% by weight, more preferably 0.5 to 20% by weight, and particularly preferably 1 to 10% by weight, based on the weight of the entire composition of the adhesive layer 3 formed.

The basic drug is only required to be a basic drug which can be administered percutaneously, examples of which include fentanyl, oxybutynin, pergolide, donepezil, ambroxol, tamsulosin, risperidone, olanzapine, tandospirone, tulobuterol and morphine. These drugs can be used in combinations of two or more types as necessary in the case problems do not occur attributable to drug interaction. Furthermore, in the present embodiment, in the case the basic drug is fentanyl, the above-mentioned molar concentration ratio [(M_{A})/(M_{B})] is preferably 1 or more, and more preferably 2 or more.

In addition, examples of pharmaceutically acceptable salts of the basic drugs include salts of the basic drugs and acids. In addition, these salts may be inorganic salts or organic salts. Specific examples include hypnotics and sedatives (such as flurazepam hydrochloride and rilmazafone hydrochloride), antifebrile, antiphlogistic analgesics (such as butorphanol tartrate and perisoxal citrate), stimulants and antihypnotics (such as methamphetamine hydrochloride and methylphenidate hydrochloride), psychoneural agents (such as chlorpromazine hydrochloride and imipramine hydrochloride), local anesthetics (such as lidocaine hydrochloride and procaine hydrochloride), diuretics (such as oxybutynin hydrochloride), skeletal muscle relaxants (such as tizanidine hydrochloride, eperisone hydrochloride and pridinol mesilate), autonomic nerve agents (such as calpronium hydrochloride and neostigmine bromide), anti-Parkinson agents (such as pergolide mesilat, trihexyphenidyl hydrochloride and amantadine hydrochloride), anti-histamines (such as clemastine fumarate and diphenhydramine tanninate), bronchodilators (such as tulobuterol hydrochloride and procaterol hydrochloride), cardiacs (such as isoprenaline hydrochloride and dopamine hydrochloride), coronary vasodilators (such as diltiazem hydrochloride and verapamil hydrochloride), peripheral vasodilators (such as nicametate citrate and tolazoline hydrochloride), circulatory agents (such as flunarizine hydrochloride and nicardipine hydrochloride), antiarrhythmics (such as propranolol hydrochloride and alprenolol hydrochloride), antiallergics (such as ketotifen fumarate and azelastine hydrochloride), antivertigo agents (such as betahistine mesilate and difenidol hydrochloride), serotonin receptor antagonistic antiemetics, and narcotic-based analgesics (such as morphine sulfate and fentanyl citrate). These drugs can be used in combinations of two or more types as necessary in the case problems do not occur attributable to drug interaction.

In the present embodiment, fentanyl, oxybutynin, pergolide or donepezil is preferably used as a basic drug and/or basic drug of a pharmaceutically acceptable salt thereof. Namely, fentanyl, oxybutynin, pergolide or donepezil, which is formed as an ion pair with an anion component (particularly, with the above-mentioned volatile organic acid), is preferably contained in the adhesive layer 3. Although previously stated, in the case the basic drug is fentanyl, the molar concentration ratio [(M_{A})/(M_{B})] is preferably 1 or more. As a result, the free form of fentanyl is not contained, while fentanyl formed as an ion pair with an anion component (particularly, with the above-mentioned volatile organic acid) is contained in the adhesive layer 3, thereby making it possible to realize the patch preparation 1 having adequately superior drug percutaneous absorption.

In consideration of being able to obtain adequate pharmacological effects and irritation of the skin during use as a patch preparation, the content of the basic drug and/or pharmaceutically acceptable salt thereof in the adhesive base is preferably set to be 0.1 to 70% by weight, more preferably 0.5 to 55% by weight, and particularly preferably 1 to 40% by weight, based on the weight of the entire composition of the adhesive layer 3 formed.

In addition, from the viewpoint of further promoting drug percutaneous absorption, the adhesive base preferably contains an organic acid or organic acid salt other than the above-mentioned volatile organic acid.

Examples of organic acids other than the volatile organic acid include aromatic carboxylic acids such as phthalic acid; alkylsulfonic acids such as ethanesulfonic acid, propylsulfonic acid, butanesulfonic acid and polyoxyethylene alkyl ether sulfonic acid; alkylsulfonic acid derivatives such as N-2-hydroxyethylpiperidine-N′-2-ethanesulfonic acid; and, cholic acid derivatives such as dehydrocholic acid.

Examples of organic acid salts include water-soluble inorganic salts of aliphatic (mono-, di- and tri-) carboxylic acids such as acetic acid, propionic acid, isobutyric acid, caproic acid, lactic acid, maleic acid, pyruvic acid, oxalic acid, succinic acid and tartaric acid; aromatic carboxylic acids such as phthalic acid, salicylic acid, benzoic acid and acetylsalicylic acid; alkylsulfonic acids such as ethanesulfonic acid, propylsulfonic acid, butanesulfonic acid and polyoxyethylene alkyl ether sulfonic acid; alkylsulfonic acid derivatives such as N-2-hydroxyethylpiperidine-N′-2-ethanesulfonic acid; and cholic acid derivatives such as dehydrocholic acid. These can be contained alone or two or more types can be contained in combination. In addition, although these organic acid salts may be in the form of anhydrides or hydrates, anhydrides are preferable in the case the adhesive layer 3 is hydrophobic.

In addition, specific examples of the organic acid salts include sodium acetate, sodium propionate, sodium lactate, trisodium citrate, sodium tartrate and sodium fumarate. Moreover, in the present embodiment, the adhesive base preferably contains one or more types of sodium acetate, sodium proprionate, sodium lactate and trisodium citrate.

In consideration of drug percutaneous absorption promoting effects and irritation of the skin, the content of the organic acid salt in the adhesive base is preferably set to be 0.1 to 30% by weight, more preferably 0.5 to 20% by weight, and particularly preferably 1 to 10% by weight, based on the weight of the entire composition of the adhesive layer 3 formed.

In addition, the adhesive base can contain an absorption promoter other than the above-mentioned organic acid salt. The absorption promoter may be a conventional compound which is recognized to have absorption promoting action in the skin, examples of which include fatty acids having 6 to 20 carbon atoms, fatty alcohols, fatty acid esters or ethers, aromatic organic acids, aromatic alcohols, aromatic organic acid esters or ethers (all of which may be saturated or unsaturated, and may be cyclic, linear or branched), lactic acid esters, acetic acid esters, monoterpene compounds, sesquiterpene compounds, azones, azone derivatives, glycerin fatty acid esters, sorbitan fatty acid esters (Span-type), polysorbates (Tween-type), polyethylene glycol fatty acid esters, polyoxyethylene hardened castor oil (HCO-type), and sucrose fatty acid esters.

Specific examples these absorption promoters include caprylic acid, capric acid, caproic acid, lauric acid, myristic acid, palmitic acid, stearic acid, oleic acid, linoleic acid, linolenic acid, lauryl alcohol, myristyl alcohol, isostearyl alcohol, oleyl alcohol, cetyl alcohol, methyl laurate, isopropyl myristate, myristyl myristate, octyldecyl myristate, cetyl palmitate, salicylic acid, methyl salicylate, ethylene glycol salicylate, cinnamic acid, methyl cinnamate, cresol, cetyl lactate, ethyl acetate, propyl acetate, geraniol, thymol, eugenol, terpineol, 1-menthol, borneol, d-limonene, isoeugenol, isoborneol, nerol, d1-camphor, glycerin monolaurate, glycerin monooleate, sorbitan monolaurate, sucrose monolaurate, polysorbate 20, propylene glycol, polyethylene glycol monolaurate, polyethylene glycol monostearate, HCO-60 and 1-[2-(decylthio)ethyl]azacyclopentan-2-one (abbreviated as pyrothiodecane). In the present embodiment, sorbitan monolaurate, pyrothiodecane, isostearyl alcohol, lauric acid diethanol amide, propylene glycol monolaurate, glycerin monolaurate, lauric acid and isopropyl myristate are preferable.

In consideration of drug percutaneous absorption promoting effects and irritation of the skin, the content of the absorption promoter in the adhesive base is preferably set to be 0.1 to 30% by weight, more preferably 0.5 to 20% by weight, and particularly preferably 1 to 10% by weight, based on the weight of the entire composition of the adhesive layer 3 formed.

The adhesive layer 3 of the patch preparation 1 of the present embodiment is formed using a liposoluble hydrophobic polymer. Examples of such hydrophobic polymers include styrene-isoprene-styrene block copolymer (abbreviated as SIS), isoprene rubber, polyisobutylene (abbreviated as PIB), styrene-butadiene-styrene block copolymer (abbreviated as SBS), styrene-butadiene rubber (abbreviated as SBR) and acrylic polymers (for example, at least two types of copolymers selected from the group consisting of 2-ethylhexylacrylate, vinyl acetate, methacrylate, methoxyethyl acrylate and acrylic acid). These hydrophobic polymers can be used alone or two or more types can be used in combination.

In addition, other examples of the hydrophobic polymers include those which can be acquired commercially. Examples of commercially available SIS include Califlex D-1111 and Califlex TR-1107 (trade names, Shell Chemical Japan), JSR5000, JSR-5002 and SR5100 (trade names, JSR Corp.), and Quintac 3421 (trade name, Zeon Corp.). Examples of commercially available SBS include Califlex TR-1101 (trade name, Shell Chemical Japan). Examples of acrylic polymers include PE-300 (trade name, Nippon Carbide Industries), Duro-Tak 87-4098, Duro-Tak 87-2194 and Duro-Tak 87-2516 (trade names, National Starch & Chemical Japan).

The content of the hydrophobic polymer in the adhesive base is preferably set to be 5 to 90% by weight, more preferably 15 to 80% by weight, and particularly preferably 25 to 70% by weight based on the weight of the entire composition of the adhesive layer 3 formed. If the content ratio of the hydrophobic polymer is less than 5% by weight, the cohesive strength of the adhesive layer tends to decrease, while if the content ratio of the hydrophobic polymer exceeds 90% by weight, release of drug tends to decrease.

In addition, the adhesive base can contain a tackifying resin or plasticizer to regulate adhesiveness.

Examples of tackifying resins include rosin and rosin derivatives such as glycerin esters of rosins, hydrogenated rosins, and glycerin esters of hydrogenated rosins and rosin pentaerythritol esters; alicyclic saturated hydrocarbon resins; aliphatic hydrocarbon resins; terpene resins; and, maleic acid resins. These can be used alone or two or more types can be used in combination.

Other examples of the tackifying resins include those which can be acquired commercially. Examples of commercially available terpene resins include Clearon P-125 (trade name, Yasuhara Chemical), examples of commercially available rosin resins include Forral 105 (trade name, Hercules), Super Ester S-100, Pinecrystal KE-311 and Pinecrystal KE-100 (trade names, Arakawa Chemical Industries), while examples of alicyclic saturated hydrocarbon resins include Arkon P-100 (trade name, Arakawa Chemical Industries).

In the present embodiment, an alicyclic saturated hydrocarbon resin, glycerin ester of a hydrogenated rosin, aliphatic hydrocarbon resin or terpene resin is used particularly preferably.

In consideration of adequate adhesive strength and irritation of the skin when peeled off during use as a patch preparation, the content of the tackifying resin in the adhesive base is preferably set to be 5 to 80% by weight, more preferably 10 to 60% by weight, and particularly preferably 20 to 40% by weight, based on the weight of the entire composition of the adhesive layer 3 formed.

Examples of plasticizers include petroleum oils such as paraffin processed oil, naphthene processed oil or aromatic processed oil; squalane and squalene; vegetable oils such as olive oil, camellia oil, castor oil, tall oil or peanut oil; dibasic acid esters such as dibutyl phthalate or dioctyl phthalate; liquid rubbers such as polybutene or liquid isoprene rubber; and diethylene glycol, polyethylene glycol, glycol salicylate, propylene glycol, dipropylene glycol and crotamiton. These can be used alone or two or more types can be used in combination.

In the present embodiment, liquid paraffin, liquid polybutene, glycol salicylate or crotamiton is used particularly preferably.

In consideration of maintaining adequate adhesive strength and cohesive strength during use as a patch preparation, the content of the plasticizer in the adhesive base is preferably set to be 5 to 60% by weight, more preferably 10 to 50% by weight, and particularly preferably 15 to 40% by weight, based on the weight of the entire composition of the adhesive layer 3 formed.

In addition, a water-soluble polymer can be contained in the adhesive base. As a result of the adhesive layer 3 formed containing a water-soluble polymer, perspiration and other moisture components produced by the skin can be absorbed, thereby making it possible to inhibit decreases in the adhesive strength of adhesive layer 3 as well as moisture and so forth, and improve the feel during use of the patch preparation 1.

Examples of water-soluble polymers include light silicic anhydride; cellulose derivatives such as carboxymethyl cellulose (CMC), sodium carboxymethyl cellulose (CMCNa), methyl cellulose (MC), hydroxypropyl methyl cellulose (HPMC), hydroxypropyl cellulose (HPC) and hydroxyethyl cellulose (HEC); and, starch derivatives (pullulan), polyvinyl alcohol (PVA), polyvinyl pyrrolidone (PVP), vinyl acetate (VA), carboxylvinyl polymer (CVP), ethyl vinyl acetate (EVA), eudragit, gelatin, polyacrylic acid, sodium polyacrylate, polyisobutylene-maleic anhydride copolymer, alginic acid, sodium alginate, carrageenan, gum arabic, tragacanth, karaya gum and polyvinyl methacrylate. These can be used alone or two or more types can be used in combination.

In addition, in the present embodiment, the water-soluble polymer is preferably a basic nitrogen-containing polymer. A polymer having a functional group such as an amino group, amide group, imino group or imide group can be used for the basic nitrogen-containing polymer. In the case the basic nitrogen-containing polymer has an amino group, the amino group may be a primary, secondary or tertiary amino group. In addition, in the case the amino group is a secondary or tertiary amino group, the substituted alkyl group may be linear or form a ring. Examples of such basic nitrogen-containing polymers include polyvinyl pyrrolidone or methyl methacrylate-butyl methacrylate-dimethylaminoethyl methacrylate copolymer (trade name: Eudragit E, Roehm).

As a result of forming the adhesive layer 3 from an adhesive base containing the above-mentioned basic nitrogen-containing polymer, the skin permeability of the drug and the physical properties of the preparation can be further improved. In the case of incorporating pergolide and/or pharmaceutically acceptable salt thereof in the adhesive base in particular, since the solubility of these drugs is improved, the basic drug formed as an ion pair with an anion component can be made to be present in the adhesive layer at a high concentration. In addition, the phenomenon of crystallization and precipitation of the drug can be more reliably prevented, thereby enabling the drug to withstand long-term storage and pharmacological effects thereof to be demonstrated continuously for a long period of time.

The content of the water-soluble polymer in the adhesive base is preferably set to be 0.5 to 30% by weight, more preferably 1 to 20% by weight, and particularly preferably 1 to 10% by weight, based on the weight of the entire composition of the adhesive layer 3 formed. If the content ratio of the water-soluble polymer is less than 0.5% by weight, it tends to be difficult to obtain the above-mentioned effects, while if the content ratio exceeds 30% by weight, adhesiveness of the adhesive layer tends to decrease.

In addition, the adhesive base can contain an antioxidant, filler, crosslinking agent, preservative or ultraviolet absorber as necessary.

Examples of antioxidants include tocopherol and ester derivatives thereof, ascorbic acid, ascorbyl stearate, nordihydroguaiaretic acid, dibutylhydroxytoluene (BHT) and butylhydroxyanisole.

Examples of fillers include calcium carbonate, magnesium carbonate, silicic acid salts such as aluminum silicate and magnesium silicate, silicic acid, barium sulfate, calcium sulfate, calcium zincate, zinc chloride and titanium chloride.

Examples of crosslinking agents include thermosetting resins such as amino resins, phenol resins, epoxy resins, alkyd resins or unsaturated polyesters, isocyanate compounds, block isocyanate compounds, organic crosslinking agents and inorganic crosslinking agents such as metals and metal compounds.

Examples of preservatives include ethyl paraoxybenzoate, propyl paraoxybenzoate and butyl paraoxybenzoate.

Examples of ultraviolet absorbers include p-aminobenzoic acid derivatives, anthranilic acid derivatives, salicylic acid derivatives, coumarin derivatives, amino acid compounds, imidazoline derivatives, pyrimidine derivatives and dioxane derivatives.

These antioxidants, fillers, crosslinking agents, preservatives and ultraviolet absorbers are incorporated in the adhesive base so that the total amount thereof is preferably 5% by weight or less, more preferably 3% by weight or less, and particularly preferably 1% by weight or less, based on the weight of the entire composition of the adhesive layer 3.

The adhesive layer 3 formed from the adhesive base containing the above-mentioned components is arranged on the backing 2. There are no particular limitations on the backing 2 used in the patch preparation 1 of the present embodiment provided it is able to backing the adhesive layer 3, and a stretchable or non-stretchable backing can be used.

Specific examples of the backing 2 include fiber sheets in the form of woven or non-woven fabrics of synthetic fibers, naturally-occurring fibers or compounded fibers thereof, such as polyurethane, polyester, polypropylene, polyvinyl acetate, polyvinylidene chloride, polyethylene, polyethylene terephthalate, aluminum sheet, Nylon, acrylic, cotton, rayon or acetate fibers, as well as fiber sheets made from compound materials consisting of these fibers and films having water vapor permeability.

From the viewpoints of safety, universality and stretchability, fiber sheets of woven or non-woven fabrics composed of polyester, polyethylene or polyethylene terephthalate are preferable, and fiber sheets of woven or non-woven fabrics composed of polyethylene terephthalate are more preferable. Even if they are thick, such fiber sheets have flexibility, easily follow the contour of the skin, and have low skin irritation. Moreover, the use of such a fiber sheet makes it possible to obtain a suitably self-supporting patch preparation.

In addition, the patch preparation 1 is provided with the release sheet 4 adhered on the adhesive layer 3. Examples of this release sheet 4 include films made of polyethylene terephthalate and other polyesters, polyvinyl chloride or polyvinylidene chloride, and laminated films made of wood-free paper and polyolefins. These release sheets are preferable since they enhance the ease of workability when peeling the release sheet 4 from the patch preparation 1 when silicone treatment is carried out on the side which contacts the adhesive layer 3.

In addition, the patch preparation of the present embodiment may be an aspect in which the above-mentioned component (B) does not substantially contain the free form of a basic drug. A patch preparation which does not substantially contain the free form of a basic drug can be produced using the solvent method described below by making the above-mentioned molar concentration ratio [(M_{A})/(M_{B})] to be 1 or more. Furthermore, in the present specification, the free form of a basic drug refers the basic form of a basic drug which is not involved in interactions such as the forming of an ion pair or a salt with an anion component also present, and which applies to the so-called Lewis definition.

The following provides an explanation of an example of a production method (solvent method) for the patch preparation 1 of the present embodiment.

First, an adhesive base is prepared for forming the adhesive layer 3. The adhesive base is obtained by dissolving or dispersing in a solvent the above-mentioned volatile organic acid, basic drug and/or pharmaceutically acceptable salt thereof, and other components (adhesive base preparation step).

Examples of solvents used include toluene, hexane, ethyl acetate, cyclohexane, heptane, butyl acetate, ethanol, methanol, xylene and isopropanol. These are preferably suitably selected according to the dissolved or dispersed components, and used alone or two or more types are used in combination. In the present embodiment, one type or a mixed solvent of two or more types of solvents selected from the group consisting of toluene, heptane, ethyl acetate, hexane and cyclohexane is particularly preferable.

Next, the prepared adhesive base is coated onto the release sheet 4 to form a coated film (coated film formation step).

Next, the adhesive layer 3 is formed by drying the coated film until the solvent is removed from the coated film (coated film drying step). Examples of methods used to dry the coated film include air drying or the use of a dryer.

Furthermore, in the present embodiment, the ratio [SA/SB] of the weight percentage SA of the volatile organic acid contained in the adhesive layer to the weight percentage SB based on the total weight of all components of the adhesive base excluding the solvent, of the volatile organic acid contained in the adhesive base is preferably 0.3 to 0.9. As a result of this ratio being within the range of 0.3 to 0.9, a patch preparation can be produced having adequately superior drug percutaneous absorption while securing adequate productivity.

Next, the backing 2 is laminated onto the adhesive layer 3 formed followed by cutting to a predetermined shape to produce the patch preparation 1.

Furthermore, it is necessary that the patch preparation 1 of the present embodiment be such that the molar concentration ratio [(M_{A})/(M_{B})] between the molar concentration (M_{A}) of the volatile organic acid component (A) and the molar concentration (M_{B}) of the basic drug component (B) in the adhesive layer 3 is 0.5 or more, and that a basic drug formed as an ion pair with an anion component be contained in the adhesive layer 3. Consequently, the patch preparation 1 having adequately superior drug percutaneous absorption is produced by adding an amount corresponding to the amount of volatile organic acid lost (volatilized) in the above-mentioned coated film formation step and coated film drying step to the amount of volatile organic acid incorporated into the adhesive base in the above-mentioned adhesive base preparation step.

The amount of volatile organic acid lost (volatilized) can be determined by actually measuring this amount.

In addition, in the case loss of the component (B) is observed after going through the coated film formation step and coated film drying step, a patch preparation 1 having adequately superior drug percutaneous absorption is produced by determining the amount of the component (B) lost in the same manner as described above, and adding the determined amount of the component (B) lost to the amount of basic drug and/or pharmaceutically acceptable salt thereof incorporated in the adhesive base in the adhesive base preparation step.

The molar concentration [M_{A}] of the volatile organic acid component (A) in the adhesive layer 3 can be measured according to, for example, the following method. First, a sample is obtained from the adhesive layer, and this sample is adequately shaken in a predetermined solvent. Continuing, a filtrate is obtained by filtering the solvent after shaking with a filter. The resulting filtrate is then analyzed by high-performance liquid chromatography (HPLC) followed by calculating the molar concentration of the volatile organic acid component in the adhesive layer 3. Furthermore, a solvent such as tetrahydrofuran is used for the above-mentioned predetermined solvent. In this case, volatile organic acid present in the form of an anion component of the basic drug formed as an ion pair with the anion component is also contained in the molar concentration of the component (A). In addition, any solvent can be used for the predetermined solvent provided it does not dissolve an organic acid salt, but does dissolve the volatile organic acid and volatile organic acid present in the form of an anion of the basic drug forming an anion component and ion pair.

In addition, the molar concentration [M_{B}] of the basic drug component (B) in the adhesive layer 3 can be measured according to, for example, the following method. First, a sample is obtained from the adhesive layer, and this sample is adequately shaken in a solvent such as tetrahydrofuran. Continuing, the solution after shaking is diluted with 50% methanol solution followed by centrifugal separation. The resulting supernatant is then analyzed by high-performance liquid chromatography (HPLC) followed by calculating the molar concentration of the basic drug component in the adhesive layer 3. In this case, since the basic drug is present in the form of a free form and salt thereof in the supernatant in addition to the basic drug formed as an ion pair with an anion component, all of these are included in the molar concentration of the component (B).

A patch preparation of the present invention can be used in an external skin patch of a pharmaceutical and so on.

### EXAMPLES

Although the following provides a more detailed explanation of the present invention by indicating examples of the present invention, the present invention is not limited to these examples, and can be altered in various ways within a range which does not deviate from the technical spirit of the present invention.

### Example 1

First, 4.0 parts by weight of fentanyl citrate, 1.0 parts by weight of sodium acetate, 0.7 parts by weight of acetic acid, 3.0 parts by weight of pyrothiodecane and 23.6 parts by weight of liquid paraffin were mixed using a mortar to obtain a mixture. Next, a solution comprising 20.0 parts by weight of styrene-isoprene-styrene block copolymer (SIS), 10.0 parts by weight of polyisobutylene (PIB) and 38.0 parts by weight of alicyclic saturated hydrocarbon resin (trade name: Arkon P-100, Arakawa Chemical Industries) dissolved in a solvent in the form of toluene was mixed with the above-mentioned mixture to prepare an adhesive base.

Next, after coating the prepared adhesive base onto a release paper to form a coated film, this coated film was allowed to stand undisturbed for 10 minutes at 80° C. followed by removing the solvent from the coated film by drying to form an adhesive layer (thickness: approximately 100 μm). Moreover, a backing made of PET was laminated onto the formed adhesive layer to produce a patch preparation.

### Example 2

First, 15.0 parts by weight of oxybutynin hydrochloride, 0.7 parts by weight of trisodium citrate, 2.0 parts by weight of acetic acid and 16.9 parts by weight of liquid paraffin were mixed using a mortar to obtain a mixture. Next, a solution comprising 27.0 parts by weight of styrene-isoprene-styrene block copolymer (SIS), 3.0 parts by weight of acrylic adhesive (trade name: Duro-Tak 87-4098, National Starch & Chemical Japan) and 36.3 parts by weight of alicyclic saturated hydrocarbon resin (trade name: Arkon P-100, Arakawa Chemical Industries) dissolved in a solvent in the form of toluene was mixed with the above-mentioned mixture to prepare an adhesive base.

Next, a patch preparation was produced in the same manner as Example 1 using the prepared adhesive base.

### Example 3

First, 15.0 parts by weight of oxybutynin hydrochloride, 0.7 parts by weight of trisodium citrate, 2.5 parts by weight of acetic acid and 16.2 parts by weight of liquid paraffin were mixed using a mortar to obtain a mixture. Next, a solution comprising 27.0 parts by weight of styrene-isoprene-styrene block copolymer (SIS), 3.0 parts by weight of acrylic adhesive (trade name: Duro-Tak 87-4098, National Starch & Chemical Japan) and 36.3 parts by weight of alicyclic saturated hydrocarbon resin (trade name: Arkon P-100, Arakawa Chemical Industries) dissolved in a solvent in the form of toluene was mixed with the above-mentioned mixture to prepare an adhesive base.

Next, a patch preparation was produced in the same manner as Example 1 using the prepared adhesive base.

### Example 4

First, 15.0 parts by weight of oxybutynin hydrochloride, 0.7 parts by weight of trisodium citrate, 6.5 parts by weight of acetic acid and 13.4 parts by weight of liquid paraffin were mixed using a mortar to obtain a mixture. Next, a solution comprising 27.0 parts by weight of styrene-isoprene-styrene block copolymer (SIS), 3.0 parts by weight of acrylic adhesive (trade name: Duro-Tak 87-4098, National Starch & Chemical Japan) and 36.3 parts by weight of alicyclic saturated hydrocarbon resin (trade name: Arkon P-100, Arakawa Chemical Industries) dissolved in a solvent in the form of toluene was mixed with the above-mentioned mixture to prepare an adhesive base.

Next, a patch preparation was produced in the same manner as Example 1 using the prepared adhesive base.

### Example 5

First, 9.0 parts by weight of pergolide mesilate, 1.0 parts by weight of sodium acetate, 4.0 parts by weight of acetic acid, 2.0 parts by weight of sorbitan monolaurate, 3.0 parts by weight of isostearyl alcohol and 18.4 parts by weight of liquid paraffin were mixed using a mortar to obtain a mixture. Next, a solution comprising 10.5 parts by weight of styrene-isoprene-styrene block copolymer (SIS), 4.5 parts by weight of acrylic adhesive (trade name: Duro-Tak 87-4098, National Starch & Chemical Japan), 40.0 parts by weight of alicyclic saturated hydrocarbon resin (trade name: Arkon P-100, Arakawa Chemical Industries) and 9.0 parts by weight of methyl methacrylate-butyl methacrylate-dimethylaminoethyl methacrylate copolymer (trade name: Eudragit E100, Degussa) dissolved in a mixed solvent of toluene and ethyl acetate (1:3 by mass ratio) was mixed with the above-mentioned mixture to prepare an adhesive base.

Next, a patch preparation was produced in the same manner as Example 1 using the prepared adhesive base.

### Example 6

First, 9.0 parts by weight of pergolide mesilate, 1.0 parts by weight of sodium acetate, 9.0 parts by weight of acetic acid, 2.0 parts by weight of sorbitan monolaurate, 3.0 parts by weight of isostearyl alcohol and 15.1 parts by weight of liquid paraffin were mixed using a mortar to obtain a mixture. Next, a solution comprising 10.5 parts by weight of styrene-isoprene-styrene block copolymer (SIS), 4.5 parts by weight of acrylic adhesive (trade name: Duro-Tak 87-4098, National Starch & Chemical Japan), 40.0 parts by weight of alicyclic saturated hydrocarbon resin (trade name: Arkon P-100, Arakawa Chemical Industries) and 9.0 parts by weight of methyl methacrylate-butyl methacrylate-dimethylaminoethyl methacrylate copolymer (trade name: Eudragit E100, Degussa) dissolved in a mixed solvent of toluene and ethyl acetate (1:3 by mass ratio) was mixed with the above-mentioned mixture to prepare an adhesive base.

Next, a patch preparation was produced in the same manner as Example 1 using the prepared adhesive base.

### Comparative Example 1

An adhesive base was prepared in the same manner as Example 1 with the exception of using acetic acid at a blending ratio of 0.15 parts by weight and using liquid paraffin at a blending ratio of 24.2 parts by weight.

Next, a patch preparation was produced in the same manner as Example 1 using the prepared adhesive base.

### Comparative Example 2

An adhesive base was prepared in the same manner as Example 3 with the exception of using acetic acid at a blending ratio of 1.0 parts by weight and using liquid paraffin at a blending ratio of 17.7 parts by weight.

Next, a patch preparation was produced in the same manner as Example 1 using the prepared adhesive base.

### Comparative Example 3

An adhesive base was prepared in the same manner as Example 5 with the exception of using acetic acid at a blending ratio of 1.0 parts by weight and using liquid paraffin at a blending ratio of 21.4 parts by weight.

Next, a patch preparation was produced in the same manner as Example 1 using the prepared adhesive base.

### Comparative Example 4

A patch preparation was produced in the same manner as Example 1 with the exception of preparing an adhesive base in the same manner as Example 1, coating the prepared adhesive base onto a release paper to form a coated film and allowing the coated film to stand undisturbed for 10 minutes at 120° C., followed by removing the solvent from the coated film by drying to form an adhesive layer (thickness: approximately 100 μm).

### Comparative Example 5

A patch preparation was produced in the same manner as Example 2 with the exception of preparing an adhesive base in the same manner as Example 2, coating the prepared adhesive base onto a release paper to form a coated film and allowing the coated film to stand undisturbed for 10 minutes at 120° C., followed by removing the solvent from the coated film by drying to form an adhesive layer (thickness: approximately 100 μm).

### Comparative Example 6

A patch preparation was produced in the same manner as Example 3 with the exception of preparing an adhesive base in the same manner as Example 3, coating the prepared adhesive base onto a release paper to form a coated film and allowing the coated film to stand undisturbed for 10 minutes at 120° C., followed by removing the solvent from the coated film by drying to form an adhesive layer (thickness: approximately 100 μm).

The concentrations of drug and volatile organic acid (acetic acid) in the adhesive layer were respectively measured by high-performance liquid chromatography (HPLC) for the patch preparations obtained in Examples 1 to 6 and Comparative Examples 1 to 6. The ratio of the molar concentration of volatile organic acid to the molar concentration of drug was determined as the molar concentration of volatile organic to drug in the adhesive layer. Those results are shown in Table 1. In addition, the respective proportions of drug and volatile organic acid present in 100 parts by weight of the adhesive layer as converted from the measured molar concentrations are also shown in Table 1. Moreover, the ratio (SA/SB) of the weight percentage SA of volatile organic acid contained in the adhesive layer to the weight percentage SB of volatile organic acid contained in the adhesive base based on the total weight of all components excluding the solvent in the adhesive base is also shown in Table 1.

[Quantification of Volatile Organic Acid]

Quantification of volatile organic acid in the form of acetic acid was carried out using the following calibration curve method.

<Preparation of Internal Standard Solution>

50 mg of fumaric acid were accurately weighed followed by addition of methanol to accurately bring to a volume of 200 mL. Next, 2 ml of this solution were accurately weighed followed by the addition of methanol to again accurately bring to a volume of 200 mL. This solution was then used as the internal standard solution.

<Preparation of Calibration Curve>

54 mg of acetic acid were weighed followed by the addition of water to accurately bring to a volume of 100 mL. 200 μL, 500 μL, 1 mL, 3 mL, 5 mL and 10 mL aliquots of this solution were then accurately weighed followed by the addition of 4 mL of tetrahydrofuran, 5 mL of the above-mentioned internal standard solution and 20 mL of methanol to each aliquot, and finally the addition of water to accurately bring to a volume of 100 mL. These solutions were then used as solutions for preparing the calibration curve. The calibration curve was prepared using these standard solutions.

<Extraction of Acetic Acid from Adhesive Layer>

20 cm² of adhesive layer was removed from the patch preparation into a flask followed by the accurate addition of 10 mL of tetrahydrofuran and shaking for 1 hour. Following shaking, the solution was filtered with a filter, and 4 mL of the resulting filtrate (liquid from which volatile organic acid had been removed by filtration) were accurately weighed followed by the addition of 5 mL of the internal standard solution and 20 mL of methanol thereto, after which water was added to bring to a final volume of 100 mL. The filtrate resulting from filtering this aqueous solution was used for the measurement sample.

<HPLC Operating Conditions>

Detector: UV absorption photometer (measuring wavelength: 210 nm)

Column: TSKgel ODS-80TsQA5 μm (4.6×250 mm)

Column temperature: Constant temperature in the vicinity of 40° C.

Mobile phase: 0.1% H₃PO₄

Flow rate: 1.0 mL/min

Injection volume: 30 μL

TABLE 1

| | | | | | Molar | | | | |
|---|---|---|---|---|---|---|---|---|---|
| | | | | | concentration | | | | |
| | | | | | ratio of | Proportion present in | | Ratio (SA/SB) of wt % | |
| | | | Blending ratio in | | volatile organic | 100 parts by weight of | | SA of volatile organic acid | Skin |
| | | | adhesive base | | acid and drug | adhesive layer | | contained in adhesive layer to wt % | perme- |
| | | | | Organic | in adhesive | | Organic | SB of volatile organic acid contained | ability |
| | | | Drug | acid | base (volatile | Drug | acid (parts | in adhesive base based on total weight | test |
| | | Organic | (parts by | (parts by | organic | (parts by | by | of all components excluding solvent | results |
| | Drug | acid | weight) | weight) | acid)/(drug) | weight) | weight) | in adhesive base | (μg/cm²/h) |
| Ex. 1 | Fentanyl | Acetic | 4 | 0.7 | 1.0 | 4 | 0.45 | 0.64 | 12.0 |
| | citrate | acid | | | | | | | |
| Comp. | Fentanyl | Acetic | 4 | 0.15 | 0.2 | 4 | 0.09 | 0.60 | 4.6 |
| Ex. 1 | citrate | acid | | | | | | | |
| Comp. | Fentanyl | Acetic | 4 | 0.7 | 0.4 | 4 | 0.16 | 0.23 | 5.4 |
| Ex. 4 | citrate | acid | | | | | | | |
| Ex. 2 | Oxybutynin | Acetic | 15 | 2.0 | 0.5 | 15 | 1.15 | 0.58 | 13.8 |
| | hydro- | acid | | | | | | | |
| | chloride | | | | | | | | |
| Ex. 3 | Oxybutynin | Acetic | 15 | 2.5 | 0.8 | 15 | 1.83 | 0.73 | 15.2 |
| | hydro- | acid | | | | | | | |
| | chloride | | | | | | | | |
| Ex. 4 | Oxybutynin | Acetic | 15 | 6.5 | 2.0 | 15 | 4.57 | 0.70 | 25.4 |
| | hydro- | acid | | | | | | | |
| | chloride | | | | | | | | |
| Comp. | Oxybutynin | Acetic | 15 | 1.0 | 0.3 | 15 | 0.69 | 0.69 | 9.3 |
| Ex. 2 | hydro- | acid | | | | | | | |
| | chloride | | | | | | | | |
| Comp. | Oxybutynin | Acetic | 15 | 2.5 | 0.2 | 15 | 0.38 | 0.15 | 8.8 |
| Ex. 5 | hydro- | acid | | | | | | | |
| | chloride | | | | | | | | |
| Ex. 5 | Pergolide | Acetic | 9 | 4.0 | 2.0 | 9 | 2.63 | 0.66 | 5.6 |
| | mesilate | acid | | | | | | | |
| Ex. 6 | Pergolide | Acetic | 9 | 9.0 | 4.5 | 9 | 5.92 | 0.66 | 8.8 |
| | mesilate | acid | | | | | | | |
| Comp. | Pergolide | Acetic | 9 | 1.0 | 0.4 | 9 | 0.53 | 0.53 | 1.2 |
| Ex. 3 | mesilate | acid | | | | | | | |
| Comp. | Pergolide | Acetic | 9 | 4.0 | 0.3 | 9 | 0.43 | 0.11 | 0.8 |
| Ex. 6 | mesilate | acid | | | | | | | |

Moreover, the skin permeability of the drugs contained in the patch preparations obtained in Examples 1 to 6 and Comparative Examples 1 to 6 was evaluated using the method described below. Those results are also shown in Table 1.

[Hairless Mouse Skin Permeability Test]

Skin was exfoliated from the backs of hairless mice and using the dermal layer side as the receptor layer side, the skin sample was attached to a flow through cell (5 cm²) around which warm water at 37° C. was circulated. The patch preparations obtained in Examples 1 to 6 and Comparative Examples 1 to 3 were applied to the horny layer side, and sampling was carried out hourly for 18 hours at the rate of 5 ml/hour (h) using physiological saline on the receptor layer. The receptor solution obtained each hour was measured for drug concentration by high-performance liquid chromatography after accurately measuring the flow volume, followed by calculation of the permeation rate per hour and determination of the skin permeation rate according to the equation indicated below.
Skin permeation rate (μg/cm²/h)={sample concentration (μg/ml)×flow volume (ml)}/preparation applied surface area (cm²)

As shown in Table 1, the patch preparations of Examples 1 to 6, in which the molar concentration ratio between the volatile organic acid and drug in the adhesive layer was 0.5 or more were confirmed to have a larger skin permeation rate of the drug and adequately superior drug percutaneous absorption as compared with the patch preparations of Comparative Examples 1 to 6 in which the molar concentration ratio was less than 0.5.

### INDUSTRIAL APPLICABILITY

According to the present invention, a patch preparation can be provided having adequately superior drug percutaneous absorption even in the case of being produced using ordinary patch preparation production methods.

1. A patch preparation comprising a backing and an adhesive layer provided on the backing, wherein
the adhesive layer contains:
(A) a volatile organic acid and,
(B) a basic drug,
and wherein in the adhesive layer, a molar concentration ratio [(M_{A})/(M_{B})] between molar concentration (M_{A}) of the component (A) and molar concentration (M_{B}) of the component (B) is 0.5 or more, and the component (B) contains a basic drug formed as an ion pair with an anion component.

2. The patch preparation according to claim 1, wherein the molar concentration ratio [(M_{A})/(M_{B})] is 1 or more.

3. The patch preparation according to claim 1, wherein the component (B) does not substantially contain a free form of a basic drug.

4. The patch preparation according to claim 1, wherein the adhesive layer is formed by removing a solvent from a coated film comprising an adhesive base containing the volatile organic acid, the basic drug and/or a pharmaceutically acceptable salt of the basic drug, and the solvent.

5. The patch preparation according to claim 4, wherein the solvent is one type of solvent selected from the group consisting of toluene, heptane, ethyl acetate, hexane and cyclohexane, or a mixed solvent of two or more types thereof.

6. The patch preparation according to claim 4, wherein a ratio (SA/SB) of the mass percentage SA of the volatile organic acid contained in the adhesive layer to the mass percentage SB of the volatile organic acid contained in the adhesive base based on the total mass of all components excluding the solvent in the adhesive base is preferably 0.3 to 0.9.

7. The patch preparation according to claim 4, wherein the adhesive base further contains an organic acid salt.

8. The patch preparation according to claim 1, wherein the component (B) contains a basic drug formed as an ion pair with an anion component which is formed from an organic acid salt and a salt of a basic drug.

9. The patch preparation according to claim 7, wherein the organic acid salt is at least one type selected from the group consisting of sodium acetate, sodium citrate, sodium propionate and sodium lactate.

10. The patch preparation according to claim 1, wherein the volatile organic acid is at least one type selected from the group consisting of acetic acid, propionic acid and lactic acid.

11. The patch preparation according to claim 1, wherein the basic drug is fentanyl, oxybutynin, pergolide or donepezil.

12. The patch preparation according to claim 1, wherein the adhesive layer contains a water-soluble polymer.

13. The patch preparation according to claim 12, wherein the water-soluble polymer is polyvinyl pyrrolidone or a basic nitrogen-containing polymer.

14. The patch preparation according to claim 13, wherein the basic nitrogen-containing polymer is a methyl methacrylate-butyl methacrylate-dimethylaminoethyl methacrylate copolymer.

15. A patch preparation comprising:
a backing, and
an adhesive layer provided on the backing and incorporating a volatile organic acid, and a basic drug and/or pharmaceutically acceptable salt of the basic drug, wherein
the total molar concentration of a volatile organic acid and a volatile organic acid derivative soluble in tetrahydrofuran contained in the adhesive layer is 0.5 times or more the molar concentration of the basic drug contained in the adhesive layer.
